(19) United States
(12) Patent Application Publication
(10) Pub. No.: US 2004/0128077 A1
(43) Pub. Date: Jul. 1, 2004

(54) Method and apparatus for following cells

(75) Inventors: Douglas J. Koebler, Irwin, PA (US); Charalambos N. Athanassiou, Athens (GR); Lei Qian, Oakmont, PA (US); Aleksandr I. Lisovich, Pittsburgh, PA (US)

Correspondence Address: Ansel M. Schwartz, Suite 304, 201 N. Craig Street, Pittsburgh, PA, 15213, US

(73) Assignee: Automated Cell, Inc.

(21) Appl. No.: 10/330,599

(22) Filed: Dec. 27, 2002

(51) Int. Cl. G06F 19/00; G01N 33/48; G01N 33/50; C12M 1/34

(52) U.S. Cl. 702/19; 435/287.1

(57) ABSTRACT

An apparatus for following cells includes an automated biochamber having a plate with at least one well in which cells are disposed in a controlled environment automatically maintained at desired conditions. The apparatus includes means for tracking individual cells in the well over time. A method for following cells includes the steps of instructing a camera by a computer to take a first image of a first well of a plate. There is the step of taking automatically with the camera the first image of the well at the instruction of the computer at a first-time. There is the step of identifying a first cell in the well with the computer. There is the step of instructing the camera by the computer to take a second image of the well. There is the step of taking automatically with the camera the second image of the well at the instruction of the computer at a second time. There is the step of linking the first cell in the first image to the first cell in the second image. A method for identifying cells in proximity to each other.

### FIELD OF THE INVENTION

The present invention relates to the tracking of individual cells over time. More specifically, the present invention relates to the automated tracking of individual cells over time disposed in the same well of a multi-well plate based on each individual cell's area and coordinates as found in images of the well taken over time.

### BACKGROUND OF THE INVENTION

The study of individual cells over time and how they react to their environment requires the ability to track the individual cells in an automated mode over time. To do this, it is required to acquire data about the individual cells over time and link each cell from one time to the next in the acquired data. Images taken with a camera provide for an excellent medium by which the data can be acquired of the cells in their environment. The images are then transferred to a computer for analysis and storage.

The analysis of these images requires the capability to automatically distinguish cells from their surroundings and to identify attributes of the cells, such as their respective area and their respective coordinates. These attributes of the cells not only provide critical information about the cells themselves, but also serve the purpose of providing characteristics of the cells that can be used to link each individual cell from one image to the next. By being able to link an individual cell from image to image, reveals even further information about the cell, such as how fast it is moving or how far it has moved, as well as the type of path it has taken over time. When considered in the context of the environment that the individual cell is exposed to, reveals a plethora of information that is acquired automatically over time about the cell and how it reacts to its environment and to the addition of gene, protein or drug stimulus to the cell.

The present invention provides for a way to track an individual cell over time, whether isolated from any other cells in an environment, or surrounded by other cells, all of which are located in the same environment. The present invention provides for this tracking preferably using images to capture data, and then using a computer in an automated fashion to analyze these images to identify attributes of the individual cells and to link the individual cells over time through the use of the sequential images that are taken of the individual cells to provide information about the cells and how they respond to their environment.

### SUMMARY OF THE INVENTION

The present invention pertains to an apparatus for following cells. The apparatus comprises an automated biochamber having a plate with at least one well in which cells are disposed in a controlled environment automatically maintained at desired conditions. The apparatus comprises means for automatically tracking individual cells in the well over time.

The present invention pertains to a method for following cells. The method comprises the steps of instructing a camera by a computer to take a first image of a first well of a plate. There is the step of taking automatically with the camera the first image of the first well at the instruction of the computer at a first-time. There is the step of identifying a first cell in the first well in the first image with the computer. There is the step of instructing the camera by the computer to take a second image of the well. There is the step of taking automatically with the camera the second image of the well at the instruction of the computer at a second time. There is the step of linking automatically the first cell in the first image to the first cell in the second image with the computer.

The present invention pertains to a method for identifying cells in proximity to each other. The method comprises the steps of instructing a camera by a computer to take a first image of a first well of a plate. There is the step of taking automatically with the camera the first image of the first well at the instruction of the computer at a first-time. There is the step of identifying a first cell in the first well in the first image with the computer. There is the step of identifying a second cell in the first well in the first image with the computer. There is the step of determining automatically that the first cell and the second cell are within a predetermined distance of each other.

### BRIEF DESCRIPTION OF THE DRAWINGS

In the accompanying drawings, the preferred embodiment of the invention and preferred methods of practicing the invention are illustrated in which:

FIG. 1 is a schematic representation of the present invention.

FIG. 2 is a flow chart of a method of the present invention.

FIGS. 3a-3h show the effect of IL-2 on T-cell motility.

FIGS. 4a and 4b show quantitative analysis of T-cell motility response to Cytokines.

FIG. 5 shows dendritic cell elongation induced by gamma-interferon.

FIG. 6 shows a proximity analysis.

FIG. 7 shows proximity analysis reveals the effect of Superantigen on T-cell-DC interaction.

FIGS. 8a, 8b and 8c show T-cell-DC interaction promotes maturation of DC as shown by morphological analysis.

FIG. 9 is a block diagram regarding the software structure.

### DETAILED DESCRIPTION

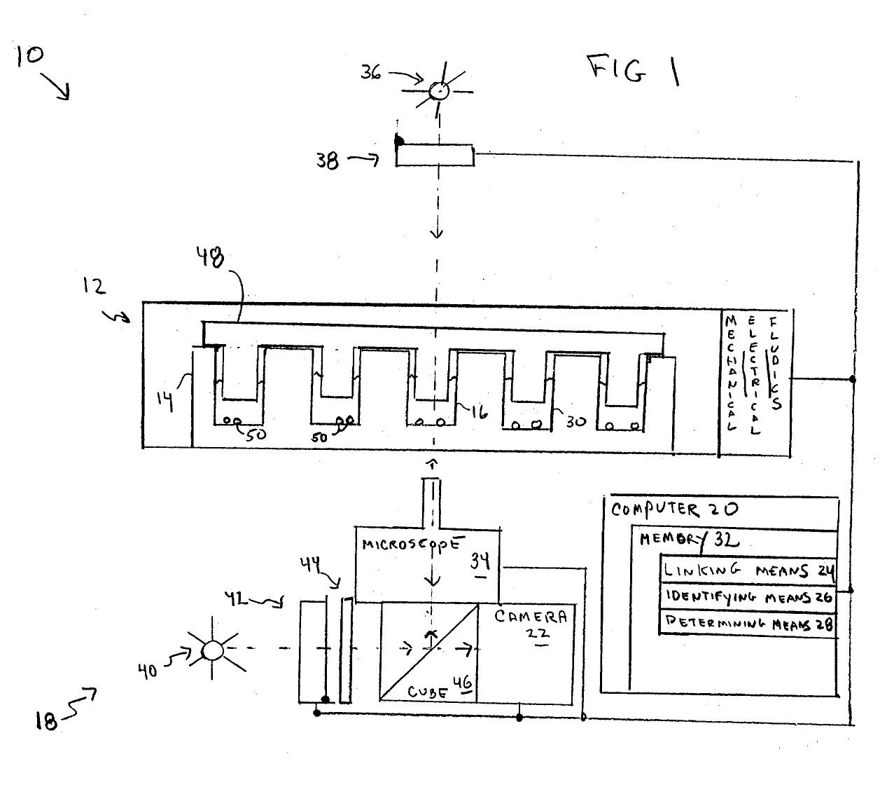

Referring now to the drawings wherein like reference numerals refer to similar or identical parts throughout the several views, and more specifically to FIG. 1 thereof, there is shown an apparatus 10 for following cells. The apparatus 10 comprises an automated biochamber 12 having a plate 14 with at least a first well 16 in which cells are disposed in a controlled environment automatically maintained at desired conditions. The apparatus 10 comprises means for automatically tracking individual cells in the first well 16 over time. It should be noted that the techniques described herein can also be performed on cells that are not disposed in a biochamber 12.

Preferably, the tracking means 18 includes a computer 20 and a camera 22 controlled by the computer 20 which takes images of the first well 16. The computer 20 preferably includes means for linking individual cells in the first well 16 in the images. Preferably, the linking means 24 includes means 26 for identifying area and coordinates of the individual cells in the first well 16 over time from the images. The linking means 24 preferably includes means 26 for determining individual cells in the first well 16 over time based on the area and the coordinates of the individual cells in the first well 16 from the images.

Preferably, the plate 14 includes a second well 30 in which cells are also disposed and the tracking means 18 automatically tracks individual cells in the second well 30 over time as well as the individual cells in the first well 16 over time. The computer 20 preferably includes a memory 32 and the linking means 24 is software disposed in the memory 32. Preferably, the tracking means 18 includes a microscope 34 in alignment with the plate 14 and the camera 22 through which the camera 22 can take images of the first well 16 and the second well 30. The microscope 34 is connected to the computer 20 and controlled by the computer 20.

Preferably, the tracking means 18 includes a visible light source 36 which illuminates the plate 14. The tracking means 18 includes a visible light source shutter 38 connected to the computer 20 which controls the visible light source shutter 38 causing the shutter to prevent visible light from illuminating the plate 14 or to allow visible light to illuminate the plate 14. The tracking means 18 includes a fluorescent light source 40 which illuminates the plate 14. The tracking means 18 includes a fluorescent light source shutter 42 connected to the computer 20 which controls the fluorescent light shutter causing the fluorescent light source shutter 42 to prevent fluorescent light from illuminating the plate 14 or allowing fluorescent light to illuminate the plate 14, The tracking means 18 includes a filter wheel 44 in alignment with the fluorescent light source 40 and the microscope 34 which controls the wavelength of the fluorescent light from the fluorescent light source 40 which illuminates the plate 14. The filter wheel 44 is made by Ludl Company, and the filters for the filter wheel 44 are made by Omega Optical Corp. A cube, also made by Omega Optical Corp., is used to direct the light from the filter wheel 44 into the microscope 34 and light from the microscope 34 coming from the plate 14 to the camera 22.

The microscope 34 is preferably inverted relative to the plate 14 so that it is disposed below the plate 14, the visible light source 36 is disposed above the plate 14, and the fluorescent light source 40 is disposed below the plate 14. Preferably, the tracking means 18 includes means 26 for determining at least two parameters from the group of parameters listed in Table 1.

The present invention pertains to a method for following cells. The method comprises the steps of instructing a camera 22 by a computer 20 to take a first image of a first well 16 of a plate 14. There is the step of taking automatically with the camera 22 the first image of the first well at the instruction of the computer 20 at a first-time. There is the step of identifying a first cell in the first well in the first image with the computer 20. There is the step of instructing the camera 22 by the computer 20 to take a second image of the well. There is the step of taking automatically with the camera 22 the second image of the well at the instruction of the computer 20 at a second time. There is the step of linking automatically the first cell in the first image to the first cell in the second image with the computer.

Preferably, the linking step includes the steps of identifying a plurality of cells in the well in the second image, and identifying the first cell in the second image from the plurality of cells in the second image. The identifying the first cell in the well step preferably includes the steps of identifying an area, shape and coordinates of the first cell in the well with the computer 20, and storing the area, shape and coordinates of the first cell in the memory 32 by the computer 20.

Preferably, the step of identifying a plurality of cells in the well includes the steps of identifying area, shape and coordinates for each of the plurality of cells in the well in the second image, and storing the area, shape and coordinates of each of the plurality of cells in the second image in the memory 32 by the computer 20. The linking step preferably includes the steps of determining the first cell in the second image based on the area, shape and coordinates of each of the plurality of cells which most closely correspond to the area, shape and coordinates of the first cell in the first image.

Preferably, there is the step of determining with the computer 20 at least two parameters from the group of parameters listed in Table 1 for the first cell based on information about the first cell stored in the memory 32. There is preferably the step of reiterating the steps of taking the second image, instructing the camera 22 to take a first image, taking, identifying, instructing the camera 22 to take a second image, and linking, for a second cell in the first well 16. Preferably, there is the step of a second step of reiterating the steps of taking the second image, instructing the camera 22 to take a first image, taking, identifying, instructing the camera 22 to take a second image, and linking, for a first cell in a second well 30. There is preferably the step of using visible light and flourescent light for taking images with the camera 22.

The present invention pertains to a method for identifying cells in proximity to each other. The method comprises the steps of instructing a camera by a computer to take a first image of a first well of a plate. There is the step of taking automatically with the camera the first image of the first well at the instruction of the computer at a first-time. There is the step of identifying a first cell in the first well in the first image with the computer. There is the step of identifying a second cell in the first well in the first image with the computer. There is the step of determining automatically that the first cell and the second cell are within a predetermined distance of each other.

Preferably the predetermined distance is 40-50 microns, or 3 or 4 cell diameters. The determining step preferably includes the step of determining automatically with a computer that the first cell has made contact with the second cell. Preferably, the determining step includes the step of determining if an object in the first image has the shape similar to a FIG. 8. The determining step preferably includes the step of associating the first cell with a cell type and the second cell with a cell type from data in a memory. Preferably, there is the step of storing in the memory that the first cell and the second cell were within the predetermined distance if the first cell and the second cell are each predetermined types of cells.

In the operation of the invention, and referring to FIG. 1, an image of a location, such as a well of a plate 14, having at least one cell and preferably a plurality of cells, is taken and stored in the memory 32 associated with a computer 20. The object identification software in the computer 20 causes the computer 20 to analyze the image to locate cells in the well.

The image is analyzed by each pixel being reviewed and having its light intensity identified. Furthermore, the relationship of the light intensity of each pixel to the pixels around it are also defined by comparing the light intensity of each pixel around a given pixel. Sharp or significant changes in pixel light intensity from one pixel to the next, indicates an edge of an object, and possibly a cell. The shape of an object is identified by connecting the pixels alongside each other, as well as the area of the object by counting how many pixels are within the object whose edge is defined by the pixels that are alongside each other and that have a significant change in pixel light intensity.

Cells are identified from the objects located by size, cells are between 10-20 microns in size, and the shape of the object. A cell generally has a roundness (an aspect ratio of 1 is round) or generally smooth curving contoured shape. If the object has sharp corners or angular features, then the object is not a cell. Objects that are identified but do not fall into this definition of a size and shape, are discarded by the computer, while those objects that meet the definition of a cell, are noted, with the coordinate location of the cell and its shape and size stored in the memory 32.

The cell that is able to be tracked or studied by the apparatus is any type of living organism like a prokaryotic or eukaryotic cell, such as animal or plant cell, including, but not limited to:

a. Single invertebrate cell

b. Single vertebrate cell

c. Single parasite organism

d. Single micro-organism (protozoan, bacterium, trypanosome, amoeba, fungus)

e. A mammalian cell, especially human, including but not limited to:

1. Muscle cell

2. Fertilized ovum

3. Glandular cell

4. Endothelial cell

5. Immunoreactive cell (T-cell, B-cell, Nk-cell, macrophage, neutrophil, basophil, mast-cell, eosinophil)

6. Hematopoeitic stem cell

7. Keratinocyte

8. Neuron or neural cell including glial cell

9. Mesenchymal cell or mesenchymal stem cell

10. Skin cell

11. Embryonal stem cell

12. Dendritic cell

f. A plant cell including but not limited to:

1. A cell from a member of the phylum angiospermae (dicotyledoneae, monocotyledmeael)

2. A cell from a member of the phylum embryophyta (gymnospermae, filicineae, hepaticae, lycopodmeae, equisetineae)

3. A cell from a member of the class chlorophyta (green algae)

Also, the cell can be protozoa, bacteria, single and multicellular organisms, as well as embryonic life forms, including fish, amphibians, reptiles, and all vertebrata. This can also apply to plant cells, as mentioned above, whether they are single-celled such as algae, slime, molds, yeasts, and other small single and multicellular organisms.

Generally speaking, any function based on unique features of the cell can be used to track a cell over time that is the choice of the user of the system. For instance, parameters found in Table 1 herein can be used to track the cell over time from one image to the next. Referring back to the features already discussed, for instance, just area and distance traveled from a previous location of the cell in the previous image can be used for tracking the cell over time in different images. An equal weighting of area verses distance can be used to identify the cell and the next image, from the previous image. If there is a tie between the function of the area and the distance between two different cells of a next image, then between these two features, the cell with the smaller distance traveled but larger area verses the larger distance traveled but smaller area dictates which cell is chosen in the next image. Another parameter or feature that can be used in addition to shape or instead of shape, can be trajectory. The assumption that would be applied is that the cell moving in a given trajectory over a previous set of images taken over a given period of time, will continue in that trajectory as applied to the next image that is taken. The trajectory is calculated from taking the coordinates identified for the cell in each image over time and connecting them to define a path that grows as later images are taken of the cell. This path and its direction yields the trajectory. These are but a few of the many possible examples of features or parameters that can be used to track a cell over images taken over time.

The tracking of a cell over time occurs by taking subsequent images of the well with the cells over time and then locating the same cell in each of the images and storing information about the cell with each image at each time the image is taken, as well as changes of the cell from image to image. Two ways that this can be accomplished are now described.

One procedure that is followed to track a cell, uses the coordinates associated with a cell obtained from an image, and causes the cross hair of the camera 22 focus to center on the coordinates of the cell the next time an image of the well; or of the cell, if there is more than one cell in the well, is taken. The coordinates of the cell identified from the image are provided to the movement program of the computer 20 that operates the movement of the well or plate 14 relative to the camera 22 to cause the camera's 22 focus to be centered on the coordinates of the cell. The subsequent image taken of the cell must occur within a short enough time span that the cell will not have moved out of the field of vision of the camera 22 from the time the image was last taken. This generally is not a problem because the types of cells that are present in the well are generally known with a sense as to how fast they may possibly move. With this knowledge, the subsequent image of a well or a cell can occur anywhere from between seconds to minutes to hours or even days from the time a previous image is taken. When a subsequent image is taken of the cell, the information obtained from the cell about the area of the cell and the distance the cell has traveled from its previous coordinates are used to confirm or link the cell in the subsequent image with the same cell in the previous image. The information about the change in shape and area of the cell, and the distance the cell has traveled is all recorded by the computer in the memory as possibly important information relative to the environment the cell is located in and its effects on the cell.

Another procedure does not require the coordinates of the cell obtained from an image to be used for the camera 22 focus for the next image. Instead, after a predetermined period of time, the automated movement system moves the well having a cell or cells relative to the camera 22 for another image to be taken of the cells in the well. When the cells are identified in the subsequent image, their attributes regarding area and distance from previous coordinates identified for the cell or cells from the previous image are compared to information about cells in the previous image. In the time that it takes for subsequent images to be taken, there is not expected to be a significant change in the area of the cell, nor an unusually large distance that the cell will have traveled. Accordingly, from the attributes regarding the area of the cell and, for instance, identifying the cell which has traveled the shortest distance from previous coordinates of a cell, the cell in the subsequent image can be linked to the same cell in the previous image.

To enhance the cells for more accurate and efficient analysis of the images, various techniques are used involving visible and fluorescent light. A well known structure uses an inverted microscope 34 with a clear multi-well plate 14, a visible light source 36 and a fluorescent light source 40, such as a mercury vapor lamp. The visible light source 36 is disposed above the plate 14 and has a shutter which, when open, allows light from the visible light source 36 to shine on the plate 14, and when closed, prevents any light from the visible light source 36 to shine on the plate 14. Below the plate 14 is the fluorescent light source 40 which is in alignment with a filter wheel 44, that is in turn in alignment with a cube 46. The filter wheel 44 allows predefined wavelengths of light from the fluorescent light source 40 to pass. The cube 46 also directs light from the fluorescent light source 40 onto the plate 14. The fluorescent light source 40 also has a shutter, which when open allows light to pass from the fluorescent light source 40 to the plate 14, and when closed prevents light from passing to the plate 14. A view path of the microscope 34 is in alignment with the path of light from the cube 46 so neither the microscope 34 nor the cube 46 block each other's view of the plate 14. A camera 22 is connected to the microscope 34 to take the images of the plate 14, and the camera's 22 focal point is the microscope's 34 focal point. Additionally, a computer 20 is connected to the camera 22, the microscope 34, the visible light shutter and the fluorescent light shutter. One of the distinctions of the present invention over the prior art is that the computer 20 controls all aspects of the image acquisition process.

The visible light from the visible light source 36 passes through the plate 14 and the cells, since the cells are translucent. Preferably, a light aligning apparatus 48 disposed in the well penetrates the meniscus of the fluid in the well to better direct the light through the well. The visible light is received by the microscope 34 objective and is ultimately received at the camera 22. In regard to the fluorescent light source 40, the cells in the well which have been treated with fluorescent material, such as with chromagen in an antibody-antigen reaction, has the fluorescent material deposited at the specific corresponding receptor sites (antigen) for the cell. Alternatively, a material such as propidium iodide is placed in the well and under certain circumstances, such as with cell death, the fluorescent material accumulates within the cell. When the fluorescent light shines on the well, the fluorescent material on or in the cells will absorb the fluorescent light at a given wavelength and emit fluorescent light at a longer wavelength than the wavelength of the light that was received. This emitted light from the cell follows a path through the microscope 34 to the cube 46, and to the camera 22.

In operation, the coordinates of the wells of the plate 14 in regard to a predetermined reference point are loaded into the computer 20. In addition, the directions for the computer 20 to follow in regard to each of the wells are loaded into the computer 20. The computer 20 then causes the various materials and cells to be introduced into the wells, if necessary, to initialize the plate 14 for the analysis.

The computer 20 then moves a well relative to the microscope 34 for analysis. The shutter to the fluorescent light is closed and the shutter to the visible light source 36 is opened (the fluorescent light source shutter 42 could instead be opened first if desired). Light from the visible light source 36 that shines on the well is received by the microscope 34. The computer 20 focuses the microscope 34 so that its focal point is at a predetermined location in the well, and an image is then taken of the well.

To assist in the focusing of the microscope 34 on the well, if desired, beads 50 can be present in the well that can be used to assist the computer 20 in focusing the microscope 34 on the well by using the beads 50 as a clear reference point, and then, for instance, backing off the focal point a given known distance from where the beads lie on the bottom of the well. Other well known techniques of autofocus can also be used.

The image is then caused to be taken by the computer 20 activating the camera 22 and the image is stored. The well can then be moved so that another well is subject to the same process while the computer 20 analyzes the image taken of the previous well. The computer 20 can cause each well in the plate 14 to be subject to the same process, unless otherwise designated, until all the wells have images taken of them and stored, after which, the well that first had an image taken of it returned to the microscope 34. By this time, the image of the well has been analyzed, and the cells have been identified that are in the view field taken by the image. Another image of the well is then taken, as explained above, and the coordinates of the cells identified in the first image along with the areas of the cells are compared to the cells identified in the second image of the well. Each cell identified in the first image is noted by the computer 20 and a search is performed on the cells identified in the second image to link the cell identified in the first image with a cell in the second image. The information about each of the cells is stored, as well as the changes, if any, to the cell and how much distance the cell has traveled, as well as the environment the cells are subjected to.

Alternatively, when each image is taken by the camera 22, instead of the well being moved away after an image of it is taken, with another well moving to take its place so its image can be taken, several images of the well can be taken. In this case, the computer 20 causes the focal point of the microscope 34 to be changed relative to the depth of the well so a focal stack of the well is formed allowing for a more accurate three dimensional analysis of the cells in the well.

The same process can be repeated with the use of fluorescent light, where the shutter to the visible light source 36 is closed. Then, information obtained through the fluorescent light analysis can also be used to obtain information about the cells in the well, and under certain circumstances, better locate the cells in the well. Alternatively, one image of the well can be taken subject to visible light, and a second image of the well can be taken with fluorescent light. In a further variation of this, instead of only one fluorescent image being taken, multiple fluorescent images can be taken with different wavelengths utilized in regard to each image. Essentially any variation of how the image or how often the image of the well as taken is available.

An illustration of the present invention is now provided. A well in a plate 14 in a controlled closed environment, such as a Biobox described in U.S. Pat. No. 6,008,010, incorporated by reference herein, having a plurality of cells is positioned in the field of view of the microscope 34. The computer 20 causes the focal point of the microscope 34 to be located at a certain point in the well. The computer 20 closes the shutter of the fluorescent light source 40 and opens the shutter of the visible light source 36 so that visible light shines on the well and is received by the microscope 34 after it has passed through the well. The computer 20 causes the camera 22 connected to the microscope 34 to take a first image the well. The computer 20 stores the image in an associated memory 32 and then analyzes the image for cells.

The computer 20 identifies a first cell in the well and stores its area, shape and coordinates in the memory 32. The computer 20 has determined that the shape of the first cell is round (having an aspect ratio of 0.95) and has an area of about 12 microns squared. The computer 20 identifies a second cell in the well and stores the area, shape and coordinates of the second cell in the memory 32. The computer 20 determines that the shape of the second cell is elliptical (having an aspect ratio of 0.45) and has an area of about 15 microns squared. The computer 20 also identifies several additional cells in the well and stores their respective areas, shapes and coordinates in the memory 32.

At a time 20 minutes after the computer 20 has taken the first image of the well, the computer 20 takes a second image of the well. As before, the computer 20 causes the focal point of the microscope 34 to be located at the certain point in the well and to receive visible light from the visible light source 36. The computer 20 stores the second image and then analyzes the second image for cells. The computer 20 identifies cells in the second image and stores their respective areas, shapes and coordinates in the memory 32.

The computer 20 then obtains from memory 32 the shape, area and coordinates of the first cell in the first image and locates the coordinates of the first cell in the first image, in the second image. The computer 20 determines that at the coordinates of the first cell in the first image, in the second image, there is no cell located, but there are several cells in the second image in proximity to the coordinates. The computer 20 stores in temporary memory 32 the shape, area and coordinates of the three cells closest to the coordinates of the first cell and the first image, that are in the second image for further analysis to identify the first cell in the second image. It should be noted that, depending on the number of cells that are within a certain distance of the coordinates in question, dictates the number of cells that will be further reviewed. In this case there are only three cells that are considered within a radius of 50 microns from the coordinates of the first cell in the first image, in the second image. The choice of 50 microns is also dependent on the circumstances, and instead can be set to any desired length, based on for instance a function of the type of cells and the environment the cells are exposed to. It should be noted that to minimize movement by the cell due to external forces, such as Brownian motion or gravity, a medium that the cells are located in the well includes a material such as methyl cellulose. With methyl cellulose present, the cells are able to also move vertically in the well, in addition to horizontally, under their own power. See PCT patent application PCT/US02/21710, incorporated by reference herein, for a more detailed discussion regarding material that minimizes the effects of external forces on cells.

The computer 20 compares the three cells that have been picked as the candidates to the first cell in the first image. If the feature of morphology or shape is used, and it does not have to be, one of the three cells has an elliptical shape with an aspect ratio of 0.54, which immediately eliminates it as a candidate to be the first cell. Of the remaining two cells, cell a has an area of 11 microns squared, an aspect ratio of 0.93, and is at a distance of 28 microns from the coordinates of the first cell in the first image, in the second image. The other remaining cell, cell b, has an area of 14 microns squared, an aspect ratio of 0.97, and is at a distance of 19 microns from the coordinates of the first cell in the first image, in the second image. The computer 20 chooses cell a to be the first cell in the second image because of the two possible choices, cell a has a size that is closest to the size of the first cell in the first image even though it is at a distance in the second image that is further from the coordinates of the first cell in the first image then cell b. The criteria that the computer 20 follows has placed a limit on how much larger a cell can be in the time period that passes between when the first image is taken and the second image is taken. In this case, there is a limitation that the cell can only change in area by 1 micron squared. Again, this can be chosen based on the circumstances and could be larger based on the type of cell and the amount of time that passes between when images are taken. The criteria further would dictate that if there are several cells that meet the criteria of size change in shape, then the cell that has traveled the least distance from the coordinates in question from the previous image, is identified as the cell that was at the coordinates in question in the previous image. An additional criteria that could be used, if necessary, is the aspect ratio cannot change more than 0.05 in 20 minutes. It should be noted the numbers used herein are for exemplary purposes. The user can use whatever numbers and limitations desired and basically dictated by the laws of physics. That is, for whatever feature is used, the change of this feature cannot be greater than the laws of physics or nature will allow. Preferably, a distribution regarding the feature is used.

If the shape is used as a feature for identifying a cell, then a subroutine should also be used. When a cell moves, such as when an adherent cell moves along the bottom surface of a well, it changes shape as it moves in a given direction. The characteristic shapes of movement can be used to further identify the cell. When a cell divides, it first rounds up into a ball, then splits. The time necessary for preparation for division, a rounded cell, as well as the identification of two rounded cells in close proximity, just after division are characteristics of a cell division. The subroutine reflects these “extreme” variations on the shape, and is only called upon when no cell is found in the new image that meets a first set of features to identify the cell in the new image. The program has data associated with these variations in terms of area and shape that is called upon when the subroutine is activated. By also taking into account time, i.e., how fast an expected event occurs, and taking images at a rate often enough to capture the event as it is occurring, the smaller the changes to the cell in the intervals between when the images are taken, and the easier (more accurate) it is to identify a given cell in a next image.

The computer 20, having chosen the cell a to be the first cell, stores the identity of cell a to be the first cell, and stores the new information about the first cell so that with respect to the second image, the first cell now has an area of 11 microns squared, an aspect ratio of 0.93, and has new coordinates, which are those identified in the second image. The computer 20 repeats this process for all the cells identified in the first image, in regard to the second image. In addition, the computer 20 repeats this process with each subsequent image taken and builds a history in regard to movement, shape and size of the first cell, and every other cell. This history can also reflect changes in the environment that have occurred to the cells at given times, which are then reflected in the images that are subsequently taken of the well. Moreover, while this example has only identified the use of visible light, as explained above, fluorescent light can be used to identify various attributes of the cell at various times in the well. For instance, at given times, the well can have propidium iodide introduced into it and then an image taken under fluorescent light to determine if any of the cells have died. The well would then be washed out of the iodide and replaced with another medium of choice with a needle pipette under the direction of the computer 20.

The data that is formed from the history of each cell can then be used to identify many different parameters associated with the cell. The following table of parameters yields additional information about the cell. From the historical data of the cell stored in memory, these parameters can be processed by the computer 20 essentially simultaneously.

TABLE 1
Parameters Measured

| | Suggested | Type of | | |
|---|---|---|---|---|
| Measurement | Name | Parameter | Description | Reference |
| 1. Colony count | Object Count | Proliferation, | The number of objects in an image, | (1-2) |
| | | apoptosis | where each object is a separated region | |
| | | | within the image outlined on the basis | |
| | | | of cell-like characteristics. | |
| 2. Object count | Cell count 1 | Proliferation, | The number of individual cells in an | |
| | | apoptosis | image, determined by dividing each | |
| | | | object area (parameter 1) by a user | |
| | | | defined average area for a cell. | |
| 3. Proliferation | Cell count 2 | Proliferation, | The number of cells in a view field, | |
| count | | apoptosis | determined by first determining the | |
| | | | average of all objects within 3 times the | |
| | | | preset preferred cell size. Then | |
| | | | dividing each colony object by that | |
| | | | average area to get a total cell count. | |
| 4. Vinst(abs) | Instantaneous | Motility | The average of the Vinst values for all | (1-2) |
| | Speed | | tracked cells in an image (see Vinst, | |
| | | | below). | |
| 5. Vinst(angle) | Instantaneous | Motility | The angle of the vector sum of the | (1-2) |
| | Direction | | displacement of the cell position | |
| | | | between the first and second points and | |
| | | | between the second and third points. | |
| 6. Vinst | Instantaneous | Motility | The vector sum of the displacement of | (1-2) |
| | Velocity | | the cell position between the first and | |
| | | | second points and between the second | |
| | | | and third points divided by the elapsed | |
| | | | time between the first and third points. | |
| 7. Vavg_inst(abs) | Instantaneous | Motility | The instantaneous speed of the average | (1-2) |
| | Smoothed | | smoothed track through a specified | |
| | Speed | | number of images before and after the | |
| | | | specific image. | |
| 8. Vavg_inst(angle) | Instantaneous | Motility | The angle of the instantaneous speed, | (1-2) |
| | Smoothed | | #7. | |
| | Angle | | | |
| 9. Vavg_inst | Average | Motility | The average of a specified number of | (1-2) |
| | Instantaneous | | images of the smoothed track at a | |
| | Velocity | | specific time/image. | |
| 10. Vsl(abs) | Straight Line | Motility | The straight-line velocity of the | (1-2) |
| | Speed | | average smoothed track. | |
| 11. Vsl(angle) | Straight Line | Motility | The angle of the straight-line velocity, | (1-2) |
| | Angle | | #10. | |
| 12. Vsl | Straight Line | Motility | The straight-line velocity of the | (1-2) |
| | Velocity | | instantaneous speeds of the track. | |
| 13. Vcl | Curvilinear | Motility | The change in the average velocity | (1-2) |
| | Speed | | over the full track up to a specific field. | |
| 14. Vavg | Average | Motility | The change in the average velocity of | (1-2) |
| | Velocity | | the smoothed track to a specific field. | |
| 15. Linearity | Linearity | Motility | The straightness of a cells motion, | (1-2) |
| | | | Vsl/Vcl. | |
| 16. Straightness | Smoothed | Motility | The same as linearity, using the | (1-2) |
| | Linearity | | smoothed track, Vsl/Vavg. | |
| 17. ALHmean | Amplitude | Motility | The measure of the oscillating | (1-2) |
| | | | amplitude of an objects motion. The | |
| | | | average amplitude of the track | |
| | | | oscillations around the smoothed track. | |
| 18. ALHmax | Maximum | Motility | The maximum amplitude of the | (1-2) |
| | Amplitude | | oscillating component of the cells | |
| | | | motion around a smoothed track. | |
| 19. BCF | Beat Cross | Motility | The average number of oscillations | (1-2) |
| | Frequency | | about the average track. | |
| 20. Circular radius | | Morphology | A measure of the circular component of | (1-2) |
| | | | the objects motion. | |
| 21. Filtered objects | | Proliferation, | The number of objects that are filtered | |
| | | apoptosis | from the analysis, based on their | |
| | | | individual speed. | |
| 22. % motile | Percent Motile | Motility | The percentage of objects that is more | (1-2) |
| | | | motile than a given area per image. | |
| 23. Elongation | Elongation | Morphology | The ratio of the length to the width of | (3) |
| (avg) | Rectangle, | | an object based upon the ratio of the | |
| | Elongation | | perimeter to the area in a rectangular | |
| | Ellipse, | | model (Elongation Rectangle) or an | |
| | Elongation | | elliptical model (Elongation Ellipse) or | |
| | Feret | | upon actual cell widths determined | |
| | | | throughout a set of angles (Elongation | |
| | | | Feret). | |
| 24. Start image | Track Segment | Experimental | The first image for which a cell | |
| | Start | | position is included in a specific track. | |
| 25. End image | Track Segment | Experimental | The final image from which a cell | |
| | End | | position was included in a specific | |
| | | | track. | |
| 26. Cyte | Cyte | Morphology | An imaging position and an associated | |
| | | | computer folder name used for | |
| | | | acquiring and storing images. | |
| 27. Avg Area | Average Area | Morphology | The average area of all the objects | (3) |
| | Pixels or | | determined from an image. | |
| | Average Area | | | |
| | Microns | | | |
| 28. Min Area | Minimum Area | Morphology | The minimum area (in pixels or | (3) |
| | Pixels or | | microns) of an object in a track or time | |
| | Minimum Area | | series. | |
| | Microns | | | |
| 29. Max Area | | Morphology | The maximum number of pixels or | (3) |
| | | | microns of an object on a track or time | |
| | | | series. | |
| 30. Mean intensity | | Morphology | The average gray scale intensity of the | (3) |
| | | | pixels within an object. | |
| 31. Intensity Sum | | Morphology | The sum of all the pixel intensities | (3) |
| | | | within an object. | |
| 32. Object Pixel | | Morphology | The standard deviation of the intensity | (3) |
| SD | | | of all the pixels within an object. | |
| 33. Area | Area Pixels or | Morphology | The number of pixels in an object or | (3) |
| | Area Square | | the area in square microns of an object. | |
| | Microns | | | |
| 34. X coord | | Motility | The x coordinate of the center of an | (3) |
| | | | object in an image. | |
| 35. Y coord | | Motility | The y coordinate of the center of an | (3) |
| | | | object in an image. | |
| 36. Perimeter | Perimeter | Morphology | The sum of the pixels around the | (3) |
| | Pixels | | perimeter of an object. | |
| 37. Fmax Diameter | | Morphology | The maximum width of an object after | (3) |
| | | | the angle is swept by a specified preset | |
| | | | angle | |
| 38. Fmin Diameter | | Morphology | The minimum width of an object after | (3) |
| | | | the angle is swept by a specified preset | |
| | | | angle. | |
| 39. Length | Length | Morphology | The maximum width of an object based | (3) |
| | Rectangle | | upon fitting the perimeter and area to a | |
| | | | rectangular model. | |
| 40. Breath | Breadth | Morphology | The minimum width of an object based | (3) |
| | Rectangle | | upon fitting the perimeter and area to a | |
| | | | rectangular model. | |
| 41. Elongation | Elongation | Morphology | The length/breath based upon fitting | (3) |
| (L/B) | Rectangle | | the perimeter and area to a rectangular | |
| | | | model. | |
| 42. Convex | | Morphology | The approximation of a convex hull of | (3) |
| Perimeter | | | an object based on a swept angle. | |
| 43. Compactness | | Morphology | The roundness of an object, perimeter | (3) |
| | | | squared/(4 pi Area). | |
| 44. Roughness | | Morphology | Measure the irregularity of the | (3) |
| | | | perimeter. Perimeter/convex | |
| | | | perimeter. | |
| 45. FElongation | Elongation | Morphology | The Fmax/Fmin. | (3) |
| | Feret | | | |
| 46. Energy | | Morphology | A measure of the variation of the | (3) |
| | | | intensity of an object. | |
| 47. Mean Energy | | Morphology | The average variation in intensity of an | (3) |
| | | | object. | |
| 48. Density | | Morphology | The accumulation of the number of | (3) |
| | | | variations divided by the area. | |
| 49. Density Sum | | Morphology | The sum of all the variations within an | (3) |
| | | | object. | |
| 50. Unique Track | | Cell-specific | A unique number for each track | |
| Index | | Delimiter | generated from cell-like objects in a | |
| | | | series of images. | |
| 51. Track Size | | Cell-specific | The length of a track in terns of the | |
| | | Delimiter- | number of cell positions included. | |
| | | Motility | | |
| 52. Track | | Cell-specific | The larger of the x or y displacements, | |
| Boundary | | Delimiter- | in pixel widths, of cell positions along | |
| (pixels) | | Motility | a track. | |
| 53. Fluorescent | | Selected | The intensity sum of an object, based | (4) |
| marker 1 | | protein | on a fluorescent marker, TRITC. | |
| | | expression | Note: Filter sets for detecting various | |
| | | marker for | fluorophore can be purchased from: | |
| | | phenotype | Chroma Technical Corp. 72 Cotton | |
| | | | Mill Hill, Unit A9 Brattleboro VT | |
| | | | 05301, U.S.A. | |
| 54. Fluorescent | | Selected | The intensity sum of an object, based | (4) |
| marker 2 | | protein | on a fluorescent marker FITC. | |
| | | expression | | |
| | | marker for | | |
| | | phenotype | | |
| 55. Fluorescent | | Selected | The intensity sum of an object, based | (4) |
| marker 3 | | protein | on a fluorescent marker DAPI. | |
| | | expression | | |
| | | marker for | | |
| | | phenotype | | |
| 56. Fluorescent | | Selected | The intensity sum of an object, based | (4) |
| marker 4 | | protein | on a fluorescent marker CY5. | |
| | | expression | | |
| | | marker for | | |
| | | phenotype | | |
| 57. Proximity (Cell | | Cell-cell | The number of cells of Type A that | |
| to cell contact) | | interactions | interact or touch a second cell of Type | |
| | | (e.g., antigen | B, based on a distance from the | |
| | | presentation) | perimeter parameter of cell Type B. | |
| 58. Frequency of | | Cell-cell | The rate of cells of Type A coming into | |
| Proximity | | interactions | proximity with a cell of Type B. | |
| | | (e.g., antigen | | |
| | | presentation) | | |
| 59. Duration of | | Cell-cell | How long the cells of Type A stay in | |
| Proximity | | interactions | contact with a cell of type B. | |
| | | (e.g., antigen | | |
| | | presentation) | | |
| 60. Cell-Specific | | Cell-cell | The number of cells interacting with a | |
| Proximity | | interactions | second cell of a specified morphology. | |
| | | (e.g., antigen | | |
| | | presentation) | | |
| 61. Phagocytosis | | Cell-cell | The number of fluorescent beads | (5) |
| Attachment | | interactions | (antigens) that are attached to a cell. | |
| | | (e.g., antigen | | |
| | | presentation) | | |
| 62. Phagocytosis | | Cell-cell | The number of fluorescent beads | (5) |
| Engulfed | | interactions | (antigens) inside a cell. | |
| | | (e.g., antigen | | |
| | | presentation) | | |
| 63. Phagocytosis | | Cell-cell | The area of fluorescent beads | (5) |
| Attachment | | interactions | (antigens) that are attached to a cell. | |
| Area | | (e.g., antigen | | |
| | | presentation) | | |
| 64. Phagocytosis | | Cell-cell | The area of fluorescent beads (5) | |
| Engulfed Area | | interactions | (antigens) inside a cell. | |
| | | (e.g., antigen | | |
| | | presentation) | | |
| 65. Persistence | | Motility | Based on the random walk model, the | |
| | | | time a cell proceeds in a given | |
| | | | direction at a consistent speed. | |

References in Table 1, all of which are incorporated by reference herein, reveal their respective parameters.
1. Knuth, U. A., Yeung, C. -H. & Nieschlag, E. (1987). Computerized semen analysis: objective measurement of semen characteristics is biased by subjective parameter setting. Fertil. Steril. 48, 118.
2. Katz, D. F. & Davis, R. O. (1987). Automatic analysis of human sperm motion. J. Androl. 8, 170.
3. Matrox Imaging Library version 5.1; User Guide; Manual No. 10513-MN-0501; Feb. 10, 1998, Matrox Electronic Systems, Ltd, Quebec, Canada.
4. Jain, R., Kasturi, R., and Schunck, B. G. Machine Vision. McGraw-Hill Book Co. Singapore, ISBN 0-07-113407-7.
5. Maniak, M., Rauchenberger, R., Albrecht, R., Murphy, J., and Gerisch, G.: Coronin involved in phagocytosis: dynamics of particle-induced relocalization visualized by a green fluorescent protein tag. Cell, 83, 915-924 (1995)

From these parameters, the effects of the environment of the well on the cells can be further determined. An example of these effects that can be identified are as follows.

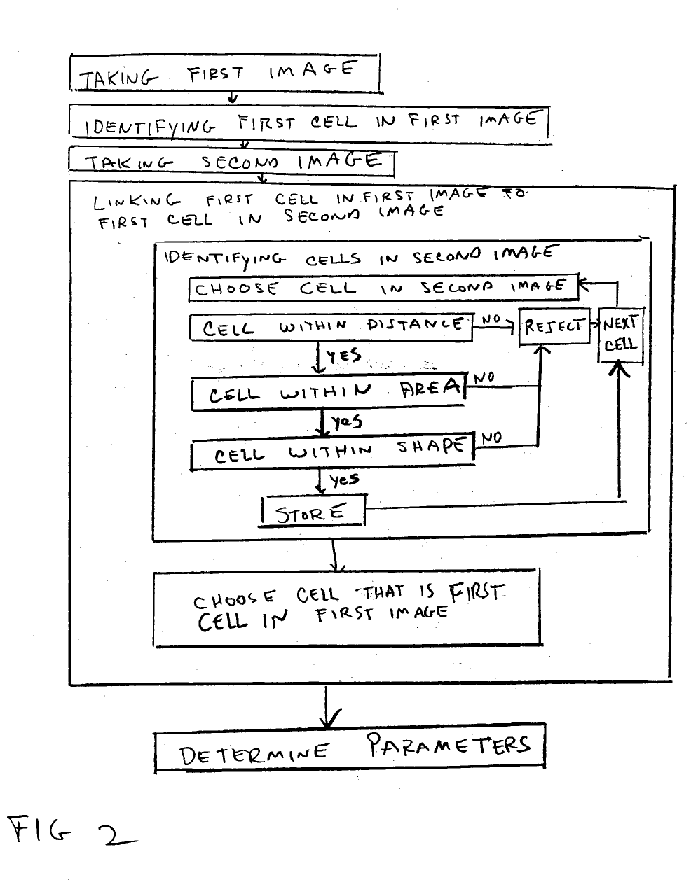

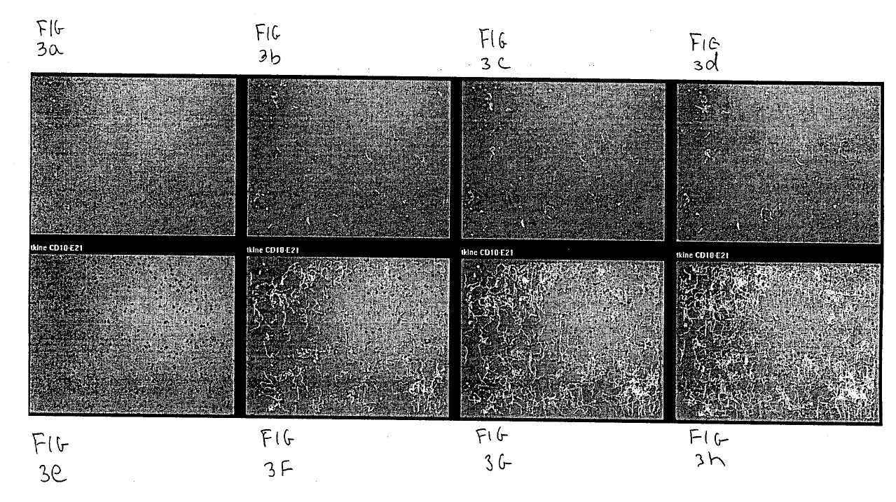

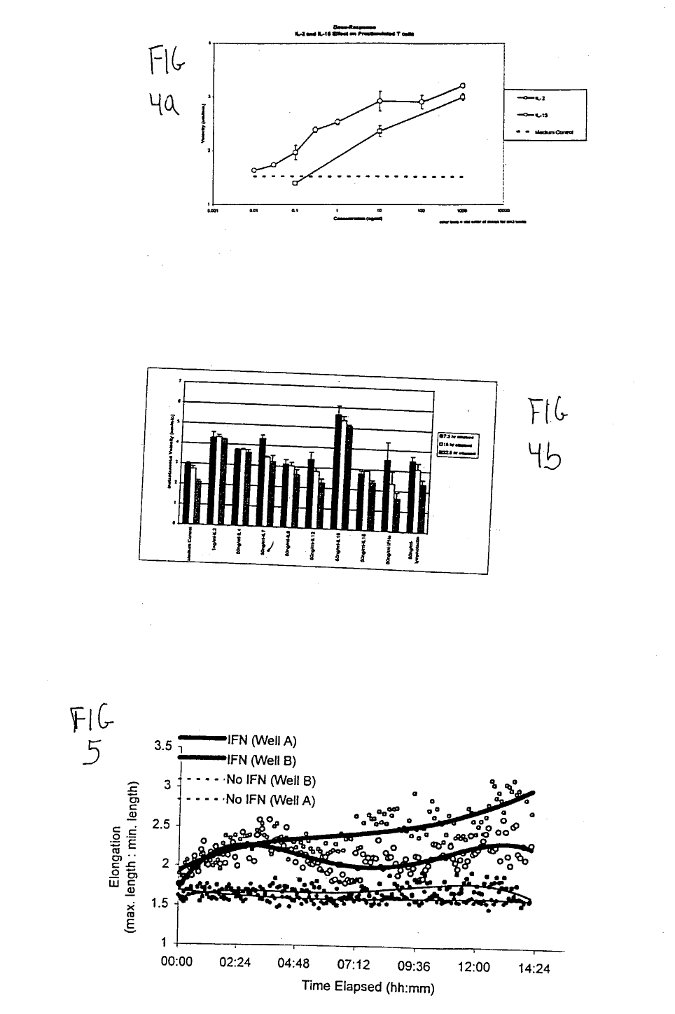

For example, the effect of IL-2 on pre-stimulated T cells is illustrated in FIGS. 3a-3h. Image sequences acquired at 2 minute intervals prior to and following addition of cytokine, chemokine or other compounds of interest are automatically processed to yield quantitative measurements for approximately 40 different parameters based upon motility, morphology, size, and texture at each time point for every individual cell in the viewfield. This view into the well is representative of the response to the compound, verified by testing multiple locations within a well.

FIGS. 3a-3h show the effect of IL-2 on T cell Motility. T cells were isolated from peripheral blood by density gradient centrifugation, monocyte depleted, stimulated for three days with phytohemagluttinin (PHA, Sigma) followed by 2 days in IL2 (5 ng/ml), washed and “rested” by overnight incubation prior to migration analysis. Shown here are processed images for T cells in the absence (top panels—green cell outlines) and presence (bottom panes—red cell outlines) of IL-2. Tracks (white lines) are built upon two-minute intervals; the 4 panels from left to right depict elapsed times of 0, 32, 64, and 96 minutes from the start of an imaging period. Dead cells have been automatically excluded from the analysis at each time point based upon propidium iodide fluorescence.

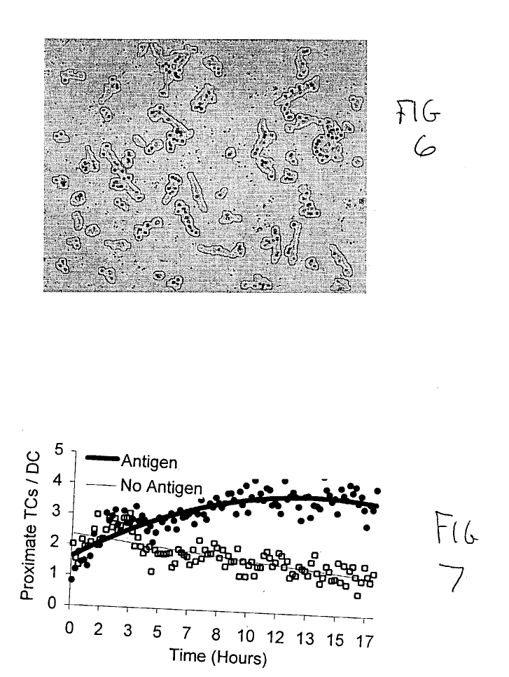

Working in a 384 well plate format enables the combinatorial cell culture system to perform, for example, dose-response studies (FIG. 4A) and to simultaneously screen multiple compounds for the magnitude and kinetics of induced effects (FIG. 4B). By cycling between groups of wells in the 384 well format, approximately 100 compounds—proteins or any chemical or biological moiety, or combinations or doses, of compounds can be analyzed simultaneously (in triplicate) to yield kinetic information over any desired time period.

FIGS. 4a and 4b show quantitative analysis of T-cell Motility Response to Cytokines. Dose-response for T cell velocity (view-field averages) in the presence of varying concentrations of IL-2 and IL-15 indicates that IL-2 induces T cell motility approximately 1.5 logs more effectively than IL-15 on a ng/ml basis (4a). Average T-cell velocity at three time points (black, white and gray bars) for each of 9 different compounds and controls illustrates capacity for screening multiple compounds simultaneously (4b). Error bars represent standard error for 3 replicate wells.

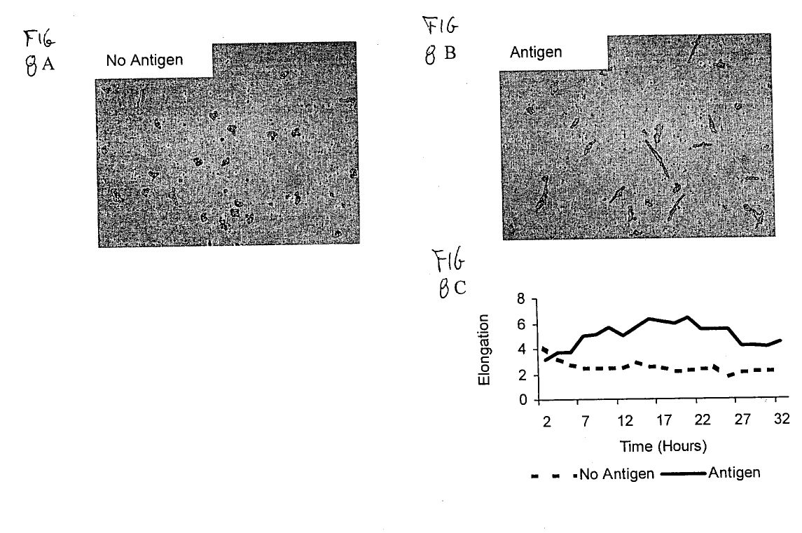

Among the morphological characteristics measured for every cell at each imaging time point is the parameter “elongation”—briefly the ratio of the length to the width. Dendritic cells (DC) are induced to elongate under the influence of gamma interferon as illustrated in FIG. 5; the kinetics of this effect is quantifiable using the combinatorial cell culture system. Heterogeneity among individual cells contributes to the high degree of variability shown by the average induced effect. While averages illustrate compound effects on the cell population as a whole, the data stored permit dissection of effects on individual cells over intervals where cells do not collide (collision frequency is a function of motility and cell density). When cell subpopulations are fluorescently labeled or otherwise distinguishable, individual cell identities may be tracked prior to, during, and subsequent to collisions (See FIG. 6).

FIG. 5 shows a Dendritic Cell Elongation is Induced by gamma-Interferon: Immature dendritic cells (DC) were generated from peripheral blood monocytes by incubation in medium containing IL-4 and GM-CSF (10001U/ml each). On day 6, loosely adherent DCs were replated into a 96 well plate in the presence (thick lines—open symbols) or absence (thin lines—closed symbols) of gamma Interferon (1000 IU/ml) and were imaged every 6 minutes. Each point represents the average elongation (length/width ratio) for 20-40 cells within each viewfield at each imaged time point. Trend lines were fitted on the data for each well (Excel—4^{th} order polynomial).

Unraveling events in coculture is not easily accomplished by methods involving bulk analysis, but imaging can provide distinguishable features that enable encounter frequencies and duration of cell-cell interactions to be quantitated. “Proximity analysis” is based upon the number of cells of one type that are located within a user defined boundary around another distinguishable cell type. The method is illustrated for DCs and T cells in FIG. 6. Distinguishing features may include differing size, morphology, motility, pixel intensity, textural qualities, or fluorescent attributes such as GFP expression or stain. Proximity analysis yields the kinetics of in vitro T cell interaction with DCs in the presence of superantigen (staphylococcus enterotoxin B (SEB)); SEB causes TCR-MHC cross linking, promotes sustained interaction, and leads to activation of T cells and maturation of DCs (FIGS. 7 and 8).

FIG. 6 shows a “Proximity Analysis”. A method for quantitation of cell-cell interaction is shown here for dendritic cells interacting with effector T cells. Dendritic cells (DC) define regions (large red outlines) within which T cells are scored as “proximate” (small red outlines). T cells outside of these regions are scored as non-proximate (small dark cells—not outlined). The percentage of proximate T cells within the view-field, or, at constant cell densities, the number of DC-proximate T cells, provide indices of interaction. More complex quantitative measures of cell-cell interaction may be derived from motility-based tracks that temporarily “terminate” or “originate” with DCs as a result of T cell-DC collisions.

FIG. 7 shows that proximity analysis reveals the effect of superantigen on T cell-DC Interaction. T cells were cocultured with immature dendritic cells in the presence (“+” marks, bold trendline) or absence (open squares, thin trendline) of staphylococcus enterotoxin B (SEB). SEB cross ligates a high percentage of TCR with the DC MHC molecules and promotes sustained interaction, the quantitation of which is evident by “proximity analysis”.

FIGS. 8a and 8b and 8c show T cell-DC interaction promotes maturation of DC as shown by morphological analysis. In the presence of SEB, T cell-DC interact ion leads to increased elongation of DCs (compare 8a and 8b). Quantitative morphological analysis reveals the kinetics of induction of this effect (8c), presumably the net result of SEB-mediated TCR-MHC class II crosslinking leading to T cell secretion of gamma interferon, inducing DC elongation (see FIG. 6).

From the above description, it can be seen that the well is representative of one continuous environment with cells (upwards of 500 cells) throughout the environment disposed in the same medium, with no solid physical structures necessarily separating them. An alternative embodiment provides that instead of the well being moved relative to the microscope 34 at various times with respect to the microscope 34 and camera 22, the well can remain stationary before the microscope 34, and with the well large enough, the microscope 34 can be moved relative to the interior of well so that different locations of the well are taken and then analyzed so that a large number of cells can be disposed in the same well and still be individually tracked. One way this can be accomplished is that a pre defined number of coordinates can be provided to the computer 20 to cause the focal point of the microscope 34 to cycle through these coordinates over time for obtaining images. The images would then be analyzed, as explained above.

Another way the well can be analyzed is by using the coordinates of cells identified in a most recently obtained image, to be the coordinates for the focal point of the microscope 34 in subsequent images. For instance, the coordinates of the first cell in a first image, can be used in a subsequent image for the focal point of the microscope 34. This could facilitate the tracking of that first cell, since the focal point can be thought of as the origin of the image relative to the cell, and was the previous coordinates of the cell in question. The analysis of the image, then occurs as described above. This could be repeated for as many cells as desired. It should be noted that all of the above embodiments are applicable to the tracking of a single cell in a single well, for an analysis of the single cell over time subject to predetermined conditions.

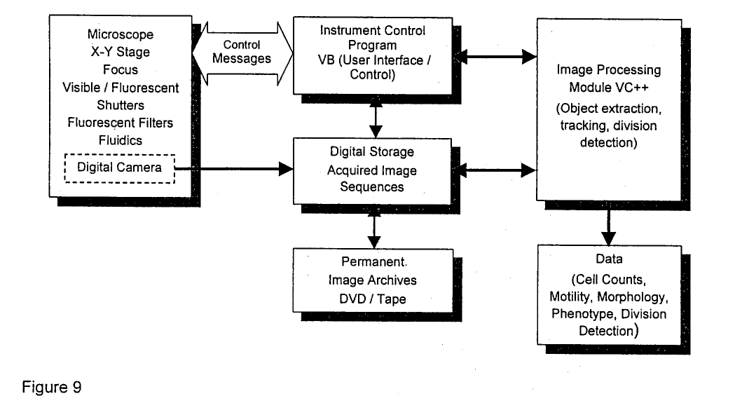

The software structure can be separated into 2 main components, Instrument Control and Image Processing (FIG. 9). The goal of the Instrument Control Program is to integrate the control of the microscope 34 stage, focus, optical filters, shutters, camera 22, fluidics, and image storing functions with cell tracking and data archiving, so that the system can record measured cell characteristics (morphology, motility, antigen expression etc.) and automatically react to programmed cell events (division, phenotype change, cell death etc.). The Instrument Control program, written in Visual Basic, utilizes the Image Processing component, written in Visual C++, through a Dynamic Link Library. This feature is employed whenever the viewfield “follows” the cell motion in real time or when event-detection is activated to trigger fluidic staining and fluorescence-based phenotyping or other functions. Otherwise, the Image Processing is used as a stand-alone program for batch processing of stored images at anytime following image acquisition. In either case, the original images are permanently stored, and reprocessing may be performed multiple times using more optimized variables or special-purpose procedures (e.g. phagocytosis analysis, dead cell “subtraction”, or cell-cell interaction analysis).

At the initiation of an experiment, the user selects the imaging locations and a list of options including whether the view-field will be moving or stationary. A moving view-field is most commonly used for single cell experiments to “follow” the cell as it migrates so that it remains in view. Immediately after acquisition, an object with features most consistent with the cell is located, and the coordinates of this object are then used as the center coordinate for the next image. The Instrument Control program controls the instrumentation to relocate and refocus upon each position in the plate 14 and return to that position at regular defined intervals, depending upon motility rate, cell density, and purpose of the experiment.

In some experiments (e.g. for dead cell subtraction using propidium iodide), fluorescent images can be acquired along with each visible image or at reduced frequencies (to reduce potential for photo-toxicity). For surface-marker phenotype analysis, single fluorescent images are acquired only after staining. Event detection based upon a change in a cell characteristic, such as cell division, cell-cell interaction or morphological change is determined through well known image processing algorithms incorporated in the Dynamic Link Library. Such events may be used, for example, to trigger fluidics staining and multi-color fluorescence image acquisition, or to change medium, add or remove compounds, or initiate changes in the nature or rate of image acquisition, if desired.

Another example of an important feature that is monitored with the apparatus is that of proximity. The feature or parameter, proximity is associated with the event of when two cells move within a given radius, or come into contact each other. This is an important phenomena in the operation of biological systems. When cells move within a certain radius of each other, or even contact each other, they are able to communicate with each other through enzymes and proteins that the cells excrete and which are received by other cells. For instance a T-cell can receive information from a dendritic cell when it moves very close or contacts the dendritic cell and receives information through the enzymes or proteins that the dendritic cell excretes and the T-cell receives. The dendritic cell can inform the T-cell about the location and existence of an alien or enemy cell. The T-cell, after receiving the information from the dendritic cell, proceeds to the alien cell, and kills the alien cell.

The apparatus provides for the ability to monitor the proximity of cells. By using the techniques described above, and including in the data that is stored, the location of the different cells identified in the image relative to each other, instances of proximity can be followed. By determining when two cells are within a given distance of each other, this would cause the two cells to be noted by the computer and that they are in proximity to each other in a given image. An additional step of identifying the type of cell each of the cells are that are in proximity to each other is also applied to determine the possibility that a communication occurred. Certain types of cells may have no ability to communicate with other types of cells. In that case, the identification of two cells within a given radius of each other, would not be considered a proximity event.

Furthermore, actual contacts that are recorded by the image of two cells can also be saved to further show that not only did the cells come within a given radius of each other, but they also contacted each other. The presence of a contact between cells can be recognized by the shape like a FIG. 8. This is because when cells come into contact with each other, they may appear to merge by the camera, since the camera may not be able to discern the boundaries of the cells when they are in contact. Instead, it appears like a FIG. 8, and this shape can be stored in the program for review and comparison to objects that are possible cells in the search or cell identification phase of the image analysis. Proximity is important in the study of chemotaxis, where it is recognized that certain types of enzymes or proteins or even cells attract or draw other cells to them. Depending on the types of cells that are being monitored, the rate at which images are taken of the cells needs to be increased or decreased so that proximity events can be captured by the images. For instance, if T-cells are being monitored, they have the ability to move very fast relative to other types of cells, and images can be taken as often as every minute or two minutes of the T-cells so proximity events involving the T-cells can be captured in the images.

When the computer wants to take an image, it first identifies the well from which the image is to be taken. From a program in memory, the computer is instructed to position the desired well in alignment with the microscope. One way this is done is by the computer instructing the drive train which moves the plate having the well to move the plate so that the well stops in a position in alignment with the microscope. This is accomplished since the computer knows the coordinates of every well in the plate. The information of all of the coordinates of the wells can be manually programmed into the memory of the computer before operation is initiated of the system, or, the computer can search out the specific coordinates of each well. For the latter to occur, when the system is first initialized, a specific location of the plate relative to the microscope is identified as the origin or reference point. The computer then goes into a search mode where it methodically causes the drive train to move the plate in predetermined increments corresponding to a field of view used to image the plate. At each incremental movement, the computer causes the camera to take an image of the plate and determines whether a well is in the image or not. This is facilitated by having the field of view of the camera and microscope be rather large so that more area can be taken in the image. By having the field of view of the camera great enough so that many wells appear in the image, the grayness scale can be used to identify the center of the wells in the image. This occurs again by the contrast of the pixels at the edge of each well will be distinct from the grayness of the pixels in the plate about the edge. The pixels having a corresponding grayness are linked to define the edge of the given well, and an approximate center of the well can be determined by calculating half the distance from the two sets of opposing sides in regard to a rectangular shaped well, or counting the pixels to determine the length of a diameter of a circle in regard to a round shaped well, and calculating the center of the circle from the diameter, as is well known in the art.

This method of locating the center of a well based on image processing preferably requires an objective smaller than 4×for a 384 well plate. A 384 well plate is 80% visible at 4×. This method works for a 1536 well plate with a 4× objective. Locating wells can occur at 4×, but then performing an autofocus at lox. The 4× image would be at 1×1 binning, (higher resolution), then blown up for locating cells. Then the operator would switch the objective to 10×, the program would do the final focus at 10×, 2×2 binning, (lower resolution), to keep the image size (file size) smaller. Alternatively, coordinates can be obtained by locating a reference point and reading in a predefined set of coordinates from the reference point, or to calculate the movement distance based on a measurement of the plate, well center to well center.

Once the plate is mapped, then the field of view can be narrowed with a corresponding increase in magnification occurring to focus on the cells in the individual wells. The edges of the plate are identified by the sharp contrast in gray scale relative to other locations in the plate. To further facilitate the mapping of the plate, the overall shape, such as a rectangle or circle, of the plate can be inputted into the computer so that once three or more points ideally are found of an edge, line segments can then be drawn by the computer. The computer can then estimate approximately where the corner or side of a plate is, and use this information as it further scans the plate and uses the line segments to identify where to expect an edge to be as it moves the plate.

Alternatively, locating and focusing on three points on a plate and then calculating a focus profile for the plate allows the plate to be realigned. This gets the operator ‘close’ to infocus for a plate that has been removed from the system then reloaded on the system for additional scans.

With the information of the location of each well in a plate, the computer instructs the drive train to move a desired well in alignment with the camera. It is generally expected that at least for the first image taken of the well, the center of the well will be used as the basis of alignment. In subsequent images of the well, depending on how cells move in the well, and which cell desires to be studied at a given time, the well can be moved in alignment with the camera so that the location of a cell in a well, obtained, for instance, from the previous image, can be used as the center point of an image of the well, which is not at the center of the well. The center of the well is also used for location for the needle, when fluidics are introduced into or out of the well.

After the well is in alignment with the camera, the computer instructs the solenoid attached to the fluorescent light shutter to close the shutter so no fluorescent light can pass through the shutter and shine on the plate. The computer also instructs the visible light shutter to open, by activating a solenoid associated with the visible light shutter so the visible light shutter is in an open position and light from the visible light source can shine on the well. The computer instructs a motor associated with the lens of the microscope, to position the focal point of the lens of the microscope at a given position in the well. The motor can be attached to a rack and pinion movement system to which the lens is also attached to cause the lens to be moved. The computer is provided with in-depth information for a given well during initialization. In the first image taken of the well, a predetermined location for the focal point, such as the middle of the well, or a small distance above the bottom of the well, can be used. Once cells are identified in the well from images obtained of the well, then subsequent images can have the lens of the microscope at different depths in the well depending on the depth of a cell being studied. An autofocus subroutine can subsequently be used to focus on a given cell and to establish the focal point of the lens when subsequent images are taken of the well by the camera.

The computer instructs a solenoid attached to the camera shutter to open the camera shutter to take the image, when the proper light is shining on the well and the focal point of the microscope lens is at a desired position. The image obtained by the camera is then stored in the memory attached to the camera for subsequent processing by the computer. Depending on the procedure chosen, such as the computer causing an image of another well to be taken, the procedure described above repeats. Instead, if an image of the same location in fluorescent light is desired, the computer instructs the visible light shutter solenoid to close the visible light shutter and instructs the fluorescent light shutter solenoid to open the fluorescent light shutter so fluorescent light shines on the well. Another image can be taken of the well at the same location but subject to visible light. If desired, instead of moving to another well, the well can be moved relative to the microscope so that a different location of the well can be taken, such as of a second cell in the well, where the second cell is then the center of an image. Alternatively, instead of moving the well relative to the microscope, the depth of the focal point of the lens in the well can be changed for an additional image. It should also be noted that the above description is based on moving the plate relative to the microscope, but the same description holds true if it is the microscope that is the motorized platform and the well is stationary, and the microscope moves relative to the plate.

U.S. Pat. No. 6,008,010, incorporated by reference herein, and PCT application PCT/US02/33813, incorporated by reference herein, describe systems that can be used as a basis to perform the operations and embodiments described herein and PCT application PCT/US02/21710, incorporated by reference herein, describes the suppression of non-biological motion of cells in wells.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

What is claimed is:

1. An apparatus for following cells comprising:
an automated biochamber having a plate with at least a first well in which cells are disposed in a controlled environment automatically maintained at desired conditions; and
means for automatically tracking individual cells in the first well over time.

2. An apparatus as described in claim 1 wherein the tracking means includes a computer and a camera controlled by the computer which takes images of the first well.

3. An apparatus as described in claim 2 wherein the computer includes means for linking individual cells in the first well in the images.

4. An apparatus as described in claim 3 wherein the linking means includes means for identifying area and coordinates of the individual cells in the first well over time from the images.

5. An apparatus as described in claim 4 wherein the linking means includes means for determining individual cells in the first well over time based on the area and the coordinates of the individual cells in the first well from the images.

6. An apparatus as described in claim 5 wherein the plate includes a second well in which cells are also disposed and the tracking means automatically tracks individual cells in the second well over time as well as the individual cells in the first well over time.

7. An apparatus as described in claim 6 wherein the computer includes a memory in the linking means is software disposed in the memory.

8. An apparatus as described in claim 7 wherein the tracking means includes a microscope in alignment with the plate and the camera through which the camera can take images of the first well and the second well, the microscope connected to the computer and controlled by the computer.

9. An apparatus as described in claim 8 wherein the linking tracking means includes a visible light source which illuminates the plate, a visible light source shutter connected to the computer which controls the visible light source shutter causing the shutter to prevent visible light from illuminating the plate or to allow visible light to illuminate the plate, a fluorescent light source which illuminates the plate, and a fluorescent light source shutter connected to the computer which controls the fluorescent light shutter causing the fluorescent light source shutter to prevent fluorescent light from illuminating the plate or allowing fluorescent light to illuminate the plate, and a filter wheel in alignment with the fluorescent light source and the microscope which controls the wavelength of the fluorescent light from the fluorescent light source which illuminates the plate.

10. An apparatus as described in claim 9 wherein the microscope is inverted relative to the plate so that it is disposed below the plate, the visible light source is disposed above the plate, and the fluorescent light source is disposed below the plate.

11. An apparatus as described in claim 10 wherein the tracking means includes means for determining at least two parameters from the group of parameters listed in Table 1.

12. A method for following cells comprising the steps of:
instructing a camera by a computer to take a first image of a first well of a plate;
taking automatically with the camera the first image of the well at the instruction of the computer at a first-time;
identifying a first cell in the first well in the first image with the computer;
instructing the camera by the computer to take a second image of the well;
taking automatically with the camera the second image of the well at the instruction of the computer at a second time; and
linking automatically the first cell in the first image to the first cell in the second image with the computer.

13. A method as described in claim 12 wherein the linking step includes the steps of identifying a plurality of cells in the well in the second image, and identifying the first cell in the second image from the plurality of cells in the second image.

14. A method as described in claim 12 wherein the identifying the first cell in the well step includes the steps of identifying an area and coordinates of the first cell in the well with the computer, and storing the area, shape and coordinates of the first cell in the memory by the computer.

15. A method as described in claim 14 wherein the step of identifying a plurality of cells in the well includes the steps of identifying area, shape and coordinates for each of the plurality of cells in the well in the second image, and storing the area, shape and coordinates of each of the plurality of cells in the second image in the memory by the computer.

16. A method as described in claim 15 wherein the linking step includes the steps of determining the first cell in the second image based on the area, shape and coordinates of each of the plurality of cells which most closely correspond to the area and coordinates of the first cell in the first image.

17. A method as described in claim 16 including the step of determining with the computer at least two parameters from the group of parameters listed in Table 1 for the first cell based on information about the first cell stored in the memory.

18. A method as described in claim 17 including the step of reiterating the steps of taking the second image, instructing the camera to take a first image, taking, identifying, instructing the camera to take a second image, and linking, for a second cell in the first well.

19. A method as described in claim 18 including a second step of reiterating the steps of taking the second image, instructing the camera to take a first image, taking, identifying, instructing the camera to take a second image, and linking, for a first cell in a second well.

20. A method as described in claim 19 including the step of using visible light and flourescent light for taking images with the camera.

21. A method for identifying cells in proximity to each other comprising the steps of:
instructing a camera by a computer to take a first image of a first well of a plate;
taking automatically with the camera the first image of the first well at the instruction of the computer at a first-time;
identifying a first cell in the first well in the first image with the computer;
identifying a second cell in the first well in the first image with the computer; and
determining automatically that the first cell and the second cell are within a predetermined distance of each other.

22. A method for taking an image of a well having a least one cell of a plate comprising the steps of:
aligning the well automatically with a computer with a microscope lens so the focal point of the lens is in the well;
closing a fluorescent light source shutter with the computer;
opening automatically with the computer a visible light source shutter so visible light shines through the well and into the lens; and
activating automatically with the computer a camera in communication with the microscope to form an image from the visible light from the well.
